(19) United States
(12) Patent Application Publication
(10) Pub. No.: US 2012/0203351 A1
(43) Pub. Date: Aug. 9, 2012

(54) EXTENDED LIFE PROSTHETIC HIP JOINT

(76) Inventor: Daniel C. Thompson, West Lebanon, NH (US)

(21) Appl. No.: 13/022,151

(22) Filed: Feb. 7, 2011

(51) Int. Cl. A61F 2/32

(52) U.S. Cl. 623/22.15

(57) ABSTRACT

A prosthetic hard-on-hard orthopaedic hip joint comprising a femoral head with a diameter of approximately 40 mm and a dimpled surface, an acetabular cup with a diameter such that the diametric clearance is less than or equal to 50 μm, a sphericity less than 10% of the diametric clearance, a composite surface roughness less than 20 nm, and a femoral stem integral to said femoral head.

### FIELD OF THE INVENTION

The present invention relates to prosthetic hip joints, and in particular, to hard-on-hard prosthetic hip joints with a hydrodynamic film.

### BACKGROUND

In 2005 there were approximately 285,000 hip replacement surgeries in the U.S., at a total cost of approximately $11 B. Of these, approximately 41,000 (14.4%) were revisions, at a cost of approximately $2 B. With an aging, active population, the number of total hip replacements is expected to rise significantly and the cost of primary hip replacements in the U.S. is projected to grow to $23 B by 2030, with the associated cost of revisions growing to $3.9 B annually. The as yet unknown cost of war injuries will add to these figures.

The average lifetime of a prosthetic hip joint is typically 10 to 15 years. The principal cause of failure is wear, and the consequences of wear (e.g., loosening of the femoral stem caused by reactions between human tissue and wear particles). More recently, because of serious wear issues with Ultra-High Molecular Weight Polyethylene (UHMWPE), the medical community has been moving from designs using cobalt-chrome femoral heads against UHMWPE acetabular cups to metal-on-metal (MoM) joint (typically cobalt-chrome) components. The results have not been entirely satisfactory. Contact between the metal surfaces creates ionic wear debris, which reacts with body tissue causing loosening of the femoral stem and tissue inflammation.

FIG. 1 illustrates the construction of a typical prosthetic hip joint, including acetabular cup 2, femoral head 4, and femoral stem 6. The various combinations of materials that are currently used to replace organic materials include MoM; Metal on UHMWPE (MoP); ceramic on metal (CoM); metal on ceramic (MoC); and ceramic on ceramic (CoC). Other material/installation options are excluded in the interest of brevity.

Nominal gaps (differences in radii) are typically greater than 50 μm, the sphericity of individual components is typically greater than 214 μm, and the composite surface roughness of the components is typically greater than 70 nm. This combination ensures that a typical, metal-on-metal joint will operate in the boundary film condition—which ensures wear and, therefore, metal ion release and/or other wear debris.

The engineering principles behind creating hydrodynamic bearings are well known, and are regularly applied to disc drives, automotive engines, and other mechanical devices. Creating a hydrodynamic film (which effectively eliminates contact between mating surfaces, hence wear) requires tight tolerances on the mating components assisted by an engineered surface. Both of these features are missing from current production prosthetic joints.

Prof. D. Dowson, of the University of Leeds (see, e.g. D. Dowson et al, “A Hip Joint Simulator Study of the Performance of Metal-on-Metal Joints,” The Journal of Arthroplasty Vol. 19 No. 8 Supp. 3 (2004), pp. 124-130), developed an analytical method for predicting the fluid film lubrication of a femoral head/acetabular cup pair. Dowson defines three ranges for L (the fluid film parameter): L<1—boundary lubrication; 1<L<3—mixed lubrication; L>3—full fluid film lubrication. L is defined as L=hₘᵢₙ/R_{ac}, where hₘᵢₙ is a minimum fluid thickness and R_{ac} is a composite surface roughness. hₘᵢₙ is a function of several factors, including mean diameter of the femoral head and acetabular cup; mean diametric clearance between the femoral head and acetabular cup; viscosity of the synovial fluid in the joint (nominally a constant); sphericity of the components; angular velocity of joint movement; and load imposed on the joint. Sphericity is defined as the p-v deviation of the measured component geometry from a best-fit perfect sphere, as established using the method of least squares.

Dowson applies his analytical model to a typical hard-on-hard (cobalt-chrome for both surfaces) hip joint. Surface roughnesses as discussed below are based on ISO 1302. Dowson's empirical model is based on the equations:

R_{ac}=composite surface roughness=sqrt(R_{ab}²+Rₐₛ²)

where

R_{ab}=femoral head arithmetic average surface roughness in nanometers,

and, where

Rₐₛ=acetabular cup arithmetic average surface roughness in nanometers,

and

hₘᵢₙ=0.929(D)^{1.42}(1000D/C+1)^{0.77}(in 10⁻³ nm)

where

D is the nominal diameter, expressed in millimeters.

C is the nominal diametric clearance, expressed in micrometers.

This analysis assumes a load of 2,500 newtons, a lubricant viscosity of 0.9 mPa s, and an angular velocity of 1.5 rad/sec.

For a typical MoM design:

D=28 mm

C=100 μm

R_{ac}=70 nm

and the predicted film thickness is:

hₘᵢₙ=8.1 nm

The resulting film parameter is:

L=hₘᵢₙ/R_{ac}=8.1/70<<1.0

Thus, it is clearly predictable that a typical prosthetic joint will operate in the boundary lubrication regime—which necessarily means that wear will occur.

Therefore there is a need for a prosthetic hip joint with a hydrodynamic film, which eliminates or minimizes the contact between joint surfaces, thus reducing wear and its associated problems.

### SUMMARY OF THE INVENTION

The present invention is a prosthetic hard-on-hard orthopaedic hip joint for humans. In its most basic form, the hip joint of the present invention includes a femoral head with a surface and diameter, an acetabular cup with a surface and diameter, a diametric clearance less than or equal to 50 μm, and a composite surface roughness.

In order to maximize L, the fluid film parameter as defined above, and ensure full fluid film lubrication to minimize contact between the femoral head and acetabular cup and therefore wear, the following conditions are achieved: The femoral head diameter is as great as possible, or approximately the diameter of an “organic” femoral head. The preferred embodiment of the present invention includes a 40 mm diameter, but the diameter may range between 20 and 50 mm to approximate different sized organic femoral heads. This is as opposed to current practice, which is typically 20 to 25 mm. The diametric clearance, which is the difference between the femoral head diameter and the acetabular cup diameter, is reduced, from the current practice of approximately 100 μm (minimum) to 50 μm or less, with a lower limit of approximately 20 μm. The sphericity tolerance of the individual components is maintained to within a fraction (10%) of the diametric clearance, or within 5 μm. The 10% fraction is preferred because at that level (1) the measurement of dimensional errors is a reasonably efficient process, and (2) establishing the impact of errors below this level on joint performance would likely be obscured by other performance parameters. Such sphericity tolerance is achievable with state-of-the art precision manufacturing equipment. The surface of one of the two wear surfaces, preferably the femoral head is engineered and modified in order to amplify the hydrodynamic effect. The preferred engineered modification is a plurality of concave dimples on the surface of the femoral head where the dimples have a depth approximately equal to the diametric clearance. Composite surface roughness, R_{ac}, is significantly reduced.

The prosthetic hip joint of the present invention is a hard-on-hard joint. Material combinations for the femoral head and acetabular cup may include any combination of metals (e.g., cobalt chrome) or ceramics (e.g., alumina). Although the present invention is directed toward a prosthetic hip joint, the improvements described herein may be applicable to prosthetics for other joints.

The femoral head is preferably 40 mm, but may be between 20 and 50 mm, which approximates the range of diameters of organic femoral heads. It is a feature of the present invention to minimize or lower the composite surface roughness, R_{ac}, which is a function of the femoral head surface roughness, R_{ab}, and the acetabular cup surface roughness, Rₐₛ, specifically the square root of the sum of their squares. The preferred method of achieving this feature is to reduce the femoral head surface roughness. R_{ac} is less than 20 nm, and preferably less than 14 nm. As discussed below, it is another feature of the present invention to include an engineered surface, preferably the femoral head surface, such as a surface including dimples. The reduced femoral head surface roughness applies to the spaces between the dimples. This is because the greatest interaction between the ball and socket occur here, and any contact/wear will occur here. It is important that the introduction of the dimples not cause perturbations of the wear surface (e.g., at their lip).

Because of the multi-directional motion of a hip joint, the preferred engineering is the inclusion of dimples in the surface, similar to a golf ball. The depth of the dimples is preferably approximately equal to the diametric clearance, but may range between ±50% of the diametric clearance, and has a total hypothetical range of between 0 and the diameter of the dimple. Experiment has shown that the use of such a dimpled surface results in a film amplification ration factor of 2.5 to 3. This factor multiplies the fluid film parameter, L, produced from other factors. Although a dimpled surface is preferred, any surface that is engineered to generate converging surfaces around a hydrodynamic bearing may be substituted. That is to say, any femoral head or acetabular cup surface that is engineered so that there are places on the femoral head surface that are biconvex with the corresponding places on the acetabular cup surface may be substituted. Furthermore, individual dimples need not be absolutely spherical. Deviations from absolute sphericity and alternative shapes, such as Gaussian shapes and paraboloids, which approximate the optimal spherical dimple yield equivalent results and may be substituted.

The acetabular cup is designed to be used with the femoral head, and therefore has a diameter larger than that of the femoral head. The diametric clearance is preferably 50 μm or less. Although it is preferred that the surface of the femoral head be engineered, the acetabular cup's surface may also be engineered, or may be engineered to the exclusion of the femoral head.

It is also preferred that the sphericity of the femoral head and acetabular cup be maintained within a fraction, preferably 10%, of the diametric clearance. The following two examples demonstrate the effect of using a femoral stem with a 40 mm diameter with different diametric clearances:

### Example 1

For a diametric clearance of 100 μm:
D=40 mm
C=100 μm
R_{ac}=14 nm (10 nm per surface)
From Dowson,
hₘᵢₙ=18 nm
thus,
L=hₘᵢₙ/14 nm=1.3>1
   The result of L=1.3 for this design would not operate in the full fluid film regime. Even with an engineered surface, which provides an amplification factor or 2.5 to 3, L is only marginally in the full fluid film regime at L=3.24 to 3.9.

### Example 2

If the diametric clearance is reduced to 50 μm:
D=40 mm
C=50 μm
R_{ac}=14 nm (10 nm per surface)
From Dowson,
hₘᵢₙ=30 nm
thus,
L=hₘᵢₙ/R_{ac}=3.4
   The result of 3.4 (without the amplification factor) puts the performance marginally into the full fluid film regime. With engineered amplification, the result is well within the full fluid regime, L=8.5 to 10.2. Therefore, the application of an engineered surface to the femoral head will result in a film thickness of 8.5 (L×2.5) or higher—well into the full fluid film regime.

The preferred prosthetic hip joint of the present invention also includes a femoral stem integral to the femoral head.

Each of the aspects as described above may be made in a range so as to make the prosthetic hip joints of the present invention adaptable to accommodate patient variables, such as body weight and size, age, level and type of activity, and the health and viscosity of the patient's synovial fluid. Therefore a range of models of the present invention will be available. The specifics of each model, such as materials, femoral head diameter, diametric clearance, sphericity, engineered surface details, will be optimized using analytical, experimental, and computational methods.

The recent evolution of deterministic polishing, combined with appropriate metrology, now enables advances in the execution of advanced, low-wear designs. The combination of small clearances, tight manufacturing tolerances, and the engineered surface enable a hydrodynamic film, whereby relative motion between the femoral head and acetabular cup generates pressure in the body's own synovial fluid, which keeps surfaces separated. The cost savings to the U.S. medical system in terms of hip revision surgeries that would not have to occur is in the billions of dollars a year. The savings in pain, suffering, and recuperation for the patients is immeasurable.

Therefore it is an aspect of the present invention to provide a prosthetic hip joint including a femoral head whose diameter approximates that of an organic hip joint.

It is a further aspect of the present invention to provide a prosthetic hip joint including a femoral head with an engineered surface.

It is a further aspect of the present invention to provide a prosthetic hip joint whose diametric clearance between the femoral head and the acetabular cup is 50 μm or less.

It is a further aspect of the present invention to provide a prosthetic hip joint with improved sphericity.

It is a further aspect of the present invention to provide a prosthetic hip joint with improved surface roughness.

It is a further aspect of the present invention to provide a prosthetic hip joint with an amplified hydrodynamic film for wear minimization.

These aspects of the present invention are not meant to be exclusive. Other features, aspects, and advantages of the present invention will be readily apparent to those of ordinary skill in the art when read in conjunction with the following description, accompanying drawings, and claims.

### BRIEF DESCRIPTION OF THE DRAWINGS

FIG. 1 is a prior art depiction of a typical prosthetic hip joint.

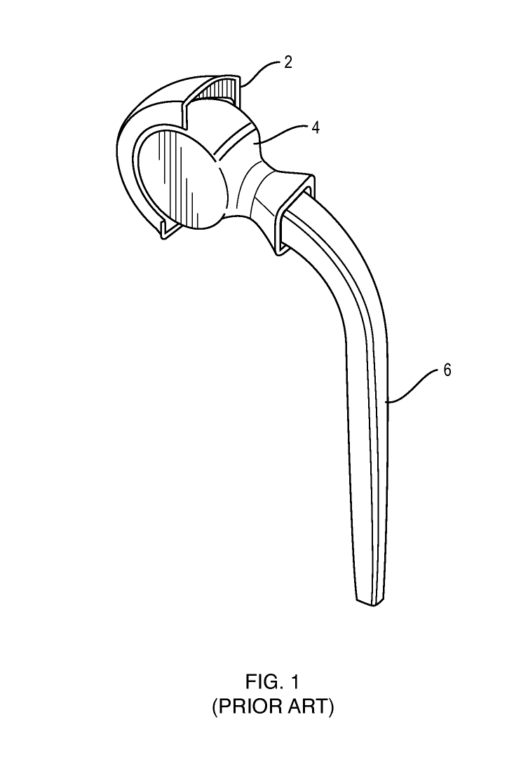

FIG. 2 is a perspective view of a prosthetic hip joint of the present invention.

FIG. 3 is a partial cutaway of the femoral head and acetabular cup of the present invention showing various measurements.

FIG. 4 is a perspective view of a preferred femoral head of the present invention.

FIG. 5 is a diagram showing a maximum dimple depth.

FIG. 6 is a diagram showing a minimum dimple depth, or no dimple.

FIG. 7 is a diagram showing a flat dimple.

### DETAILED DESCRIPTION

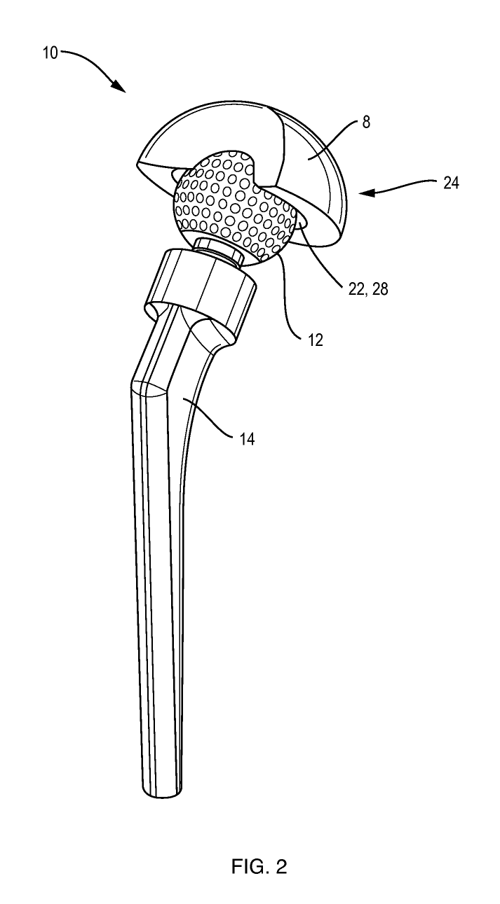

FIG. 2 is perspective view of prosthetic hip joint 10 of the present invention. In this view, we see acetabular cup 8 with acetabular cup surface 22, acetabular cup surface roughness 28, femoral head 12, femoral stem 14, and sphericity 24.

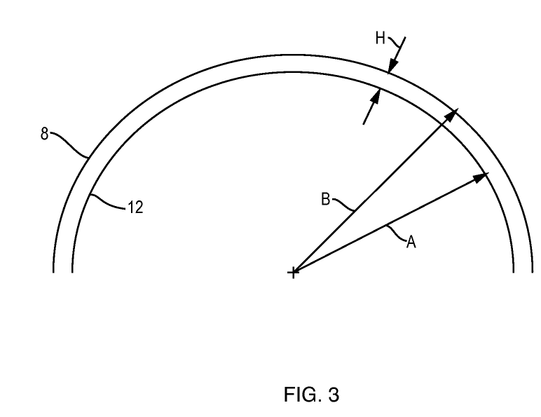

FIG. 3 is a partial cutaway of femoral head 12 and acetabular cup 8 of the present invention. Femoral head radius A is half of the femoral head diameter. Acetabular cup radius B is half of the acetabular cup diameter. Radial clearance H is the difference between A and B, and is half of the diametric clearance.

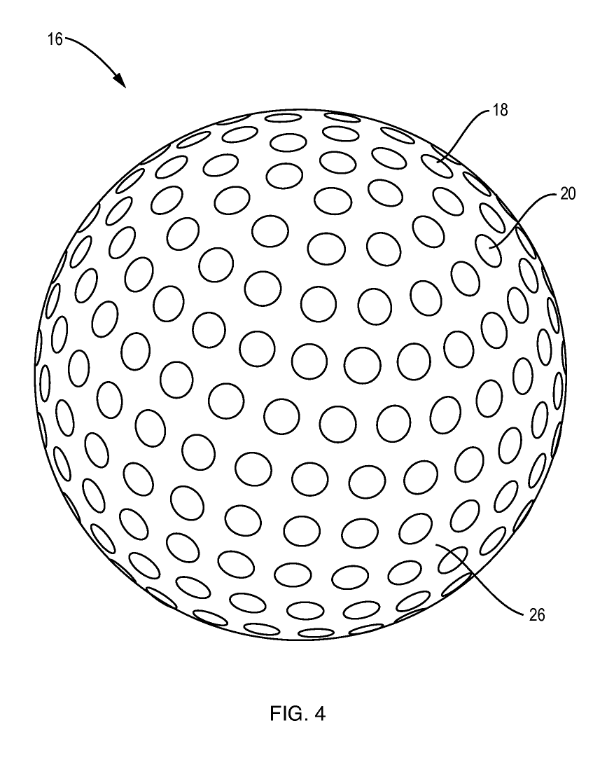

FIG. 4 is a perspective view of preferred femoral head 12 of the present invention, including dimples 18. Dimples 18 are engineered into femoral head surface 16 in a pattern similar to a golf ball. Dimples 18 have depth 20. Although not visible from this view, depth 20 is a measure of the indentation of dimple 18 into femoral head surface 16, away from the viewer. Depth 20 is preferably approximately equal to 2×H, or the diametric clearance. FIG. 4 also shows femoral head surface roughness 26, which is less than 20 nm, and preferably less than 14 nm.

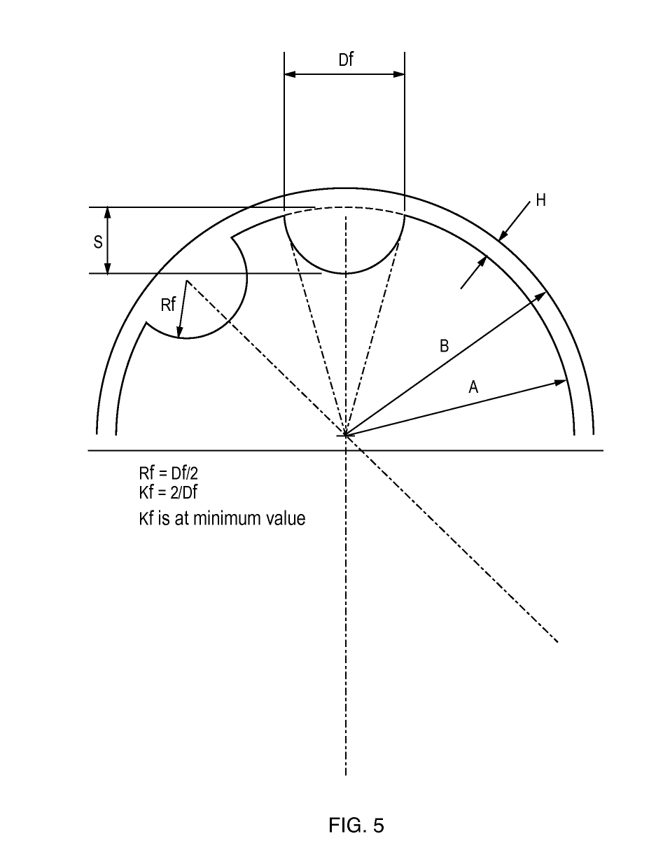

FIGS. 5-7 illustrate the general shape and geometry (radius, depth, diameter) limitations of dimple designs. The FIGS. also include descriptions of the overall geometry of the ball/socket combination (e.g., the defined bearing clearance). Although it is preferred that depth 20 approximate the diametric clearance, as discussed above, FIGS. 5-7 demonstrate the full theoretical range of depth 20.

The following terms are used below in the demonstration:

A is the femoral head radius, as shown above with reference to FIG. 3.

D_{f} is the diameter of the dimple

R_{f} is the radius of the dimple, or depth 20 as defined above.

s is the sagittal depth of the dimple, i.e. the distance between the femoral head surface and the point on the dimple's surface farthest from the femoral head surface.

H is the radial clearance as defined above with reference to FIG. 3.

K_{f} is the reciprocal of R_{f}. K_{f} is negative if the floor is concave, zero if the floor is flat, and positive if the floor is convex. K_{f} has the range −2/D_{f}≦K_{f}≦1/A.

At its minimum K_{f}=−D_{f}/2, as shown in FIG. 5. In this scenario, the dimple depth is at its maximum, with the dimple being a hemisphere.

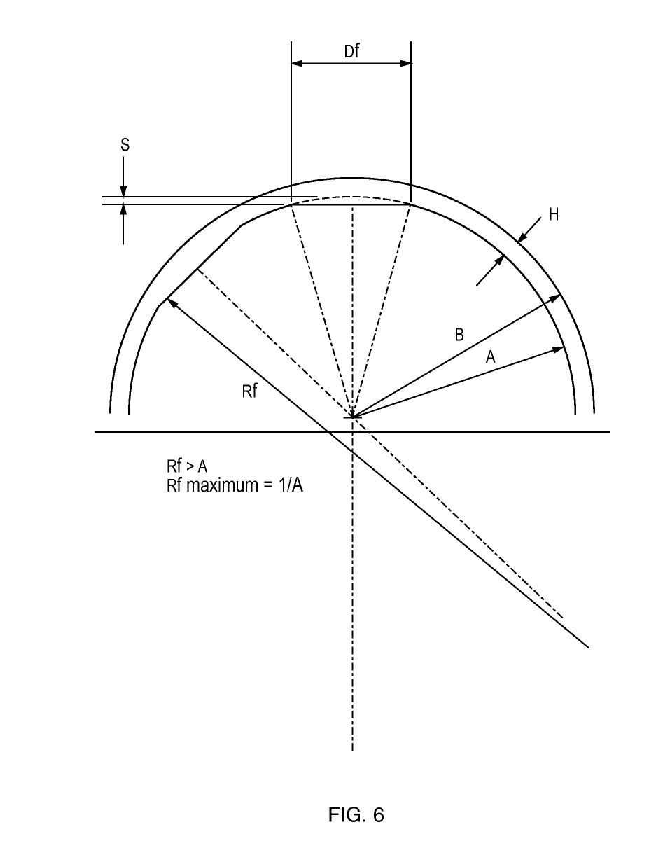

At its maximum, K_{f}=1/A, as shown in FIG. 6. In this scenario, in effect, there is no dimple, the dimple radius being equal to the femoral head radius.

A special case is one where R_{f} is set to infinity, yielding the result:

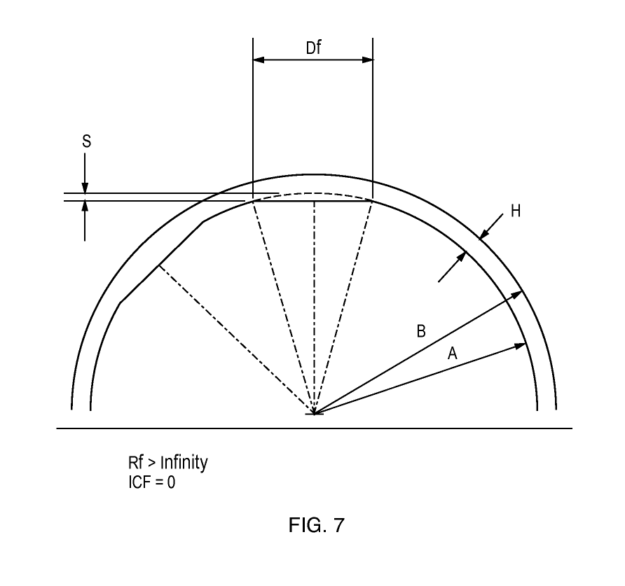

K_{f}=0, a flat, as shown in FIG. 7.

Thus a dimple array on a femoral head having radius A may be defined by specifying A, R_{f}, and D_{f} and calculating s and K_{f}. Alternatively, s, A, and K_{f} can be specified, from which D_{f} and R_{f} can be calculated. In practice, it is predicted that the designer of an individual ball/socket pair would define A, R_{f}, s, and H, thus allowing D_{f} to be calculated, and from that calculating the distribution of dimples on the surface of the ball.

Although the present invention has been described in considerable detail with reference to certain preferred versions thereof, other versions would be readily apparent to those of ordinary skill in the art. Therefore, the spirit and scope of the description should not be limited to the description of the preferred versions contained herein.

We claim:

1. A prosthetic hard-on-hard orthopaedic hip joint comprising:
a femoral head comprising a femoral head diameter dimensioned to approximate a diameter of an organic femoral head and a femoral head surface having a femoral head surface roughness;
an acetabular cup comprising an acetabular cup diameter and an acetabular cup surface having an acetabular cup surface roughness, wherein said acetabular diameter is greater than said femoral head diameter;
a diametric clearance between said femoral head surface and said acetabular cup surface that is no greater than 50 μm; and
wherein said femoral head surface and said acetabular cup surface comprise a minimized composite surface roughness, wherein said composite surface roughness is a square root of the sum of the squares of said femoral head surface roughness and said acetabular cup surface roughness.

2. The prosthetic hard-on-hard orthopaedic hip joint as claimed in claim 1, wherein said femoral head diameter is in a range of 20 to 50 mm.

3. The prosthetic hard-on-hard orthopaedic hip joint as claimed in claim 1, wherein said femoral head diameter is substantially 40 mm.

4. The prosthetic hard-on-hard orthopaedic hip joint as claimed in claim 1, wherein said femoral head and said acetabular cup each have a sphericity no greater than 10% of said diametric clearance.

5. The prosthetic hard-on-hard orthopaedic hip joint as claimed in claim 1, wherein said femoral head surface is an engineered surface shaped and dimensioned to amplify a hydrodynamic effect between said femoral head surface and said acetabular cup surface.

6. The prosthetic hard-on-hard orthopaedic hip joint as claimed in claim 5, wherein said engineered femoral head surface comprises a plurality of dimples.

7. The prosthetic hard-on-hard orthopaedic hip joint as claimed in claim 6, wherein said plurality of dimples comprise a depth approximately equal to said diametric clearance.

8. The prosthetic hard-on-hard orthopaedic hip joint as claimed in claim 5, wherein said plurality of dimples comprise a depth and a diameter, wherein a reciprocal of said depth of said plurality of dimples is no less than −2 divided by said diameter of said plurality of dimples, and wherein the reciprocal of said depth of said plurality of dimples is no greater than a reciprocal of one half of said femoral head diameter.

9. The prosthetic hard-on-hard orthopaedic hip joint as claimed in claim 1, wherein said femoral head surface is an engineered surface shaped and dimensioned to create at least one area of biconvexity between said femoral head surface and said acetabular cup surface.

10. The prosthetic hard-on-hard orthopaedic hip joint as claimed in claim 1, wherein said acetabular cup surface is an engineered surface shaped and dimensioned to amplify a hydrodynamic effect between said femoral head surface and said acetabular cup surface.

11. The prosthetic hard-on-hard orthopaedic hip joint as claimed in claim 10, wherein said engineered acetabular cup surface comprises a plurality of dimples.

12. The prosthetic hard-on-hard orthopaedic hip joint as claimed in claim 11, wherein said plurality of dimples comprise a depth approximately equal to said diametric clearance.

13. The prosthetic hard-on-hard orthopaedic hip joint as claimed in claim 1, further comprising a femoral stem extending from and integral to said femoral head.

14. The prosthetic hard-on-hard orthopaedic joint as claimed in claim 1, wherein said composite surface roughness is less than 20 nm.

15. A prosthetic hard-on-hard orthopaedic hip joint comprising:
a femoral head comprising a femoral head diameter of substantially 40 mm and a femoral head surface having a femoral head surface roughness, wherein said femoral head surface is an engineered surface shaped and dimensioned to include a plurality of dimples and wherein each of said plurality of dimples comprises a depth;
an acetabular cup comprising an acetabular cup diameter and an acetabular cup surface having an acetabular cup surface roughness, wherein said acetabular diameter is greater than said femoral head diameter;
a diametric clearance between said femoral head surface and said acetabular cup surface that is no greater than 50 μm, wherein said diametric clearance is a difference between said femoral head diameter and said acetabular cup diameter, and wherein said depth of said plurality of dimples is approximately equal to said diametric clearance;
a sphericity of said femoral head and said acetabular cup, wherein said sphericity is no greater than 10% of said diametric clearance;
wherein said femoral head surface and said acetabular cup surface comprise a composite surface roughness less than 20 nm, wherein said composite surface roughness is a square root of the sum of the squares of said femoral head surface roughness and said acetabular cup surface roughness; and
a femoral stem extending from and integral to said femoral head.

16. A prosthetic femoral head comprising a femoral head diameter dimensioned to approximate a diameter of an organic femoral head and a femoral head surface having a femoral head surface roughness wherein said femoral head surface is an engineered surface shaped and dimensioned to amplify a hydrodynamic effect between said femoral head surface and an acetabular cup surface.

17. The prosthetic femoral head as claimed in claim 16, wherein said femoral head diameter is in a range of 20 to 50 mm.

18. The prosthetic femoral head as claimed in claim 16, wherein said femoral head diameter is substantially 40 mm.

19. The prosthetic femoral head as claimed in claim 16, wherein said engineered femoral head surface comprises a plurality of dimples.

20. The prosthetic femoral head as claimed in claim 19, wherein said plurality of dimples comprise a depth and a diameter, wherein a reciprocal of said depth of said plurality of dimples is no less than −2 divided by said diameter of said plurality of dimples, and wherein the reciprocal of said depth of said plurality of dimples is no greater than a reciprocal of one half of said femoral head diameter.

21. The prosthetic femoral head as claimed in claim 16, wherein said femoral head surface is an engineered surface shaped and dimensioned to create at least one area of biconvexity between said femoral head surface and an acetabular cup surface.
